(19) United States
(12) Patent Application Publication
(10) Pub. No.: US 2026/0349513 A1
(43) Pub. Date: Nov. 5, 2026

(54) ELECTRICAL STIMULATION FOR HAPTIC FEEDBACK

(71) Applicant: Oura Health Oy, Oulu (FI)

(72) Inventor: Edoardo D'Anna, San Diego, CA (US)

(21) Appl. No.: 19/468,870

(22) Filed: Feb. 3, 2026

Related U.S. Application Data
(60) Provisional application No. 63/753,820, filed on Feb. 4, 2025.

(51) Int. Cl. G06F 3/01; G06F 1/16

(52) U.S. Cl. CPC G06F 3/016; G06F 1/163; G06F 3/015

(57) ABSTRACT

Methods, systems, and devices for electrical stimulation for haptic feedback are described. A wearable ring device may include an outer housing, an inner housing, and a set of electrodes positioned at least partially within the inner housing. The set of electrodes may include a first electrode and a second electrode, and a position of the first electrode, a position of the second electrode, or both, may be based on a size of the wearable ring device. The wearable ring device may include circuitry electrically coupled with the set of electrodes and configured to generate a current between the first electrode and the second electrode via an electrical path external to the wearable ring device. The circuitry may be further configured to modulate a frequency of the current, and the frequency may be configured for electric current-based haptic feedback.

### CROSS REFERENCE

The present Application for Patent claims the benefit of U.S. Provisional Patent Application No. 63/753,820 by D'ANNA et al., entitled “ELECTRICAL STIMULATION FOR HAPTIC FEEDBACK,” filed Feb. 4, 2025, assigned to the assignee hereof, and expressly incorporated by reference herein.

### FIELD OF TECHNOLOGY

The following relates to wearable devices and data processing, including electrical stimulation for haptic feedback.

### BACKGROUND

Some wearable devices may be configured to provide feedback to users via alerts or other notifications, where the alerts or other notifications may communicate information about the health and well-being of the user, or may communicate operating parameters of the wearable ring device, such as battery information or device settings. The feedback may be provided to the user via various communicative means, such as by using lights, sounds, or vibrations, which may vary in their effectiveness and ease of use.

### BRIEF DESCRIPTION OF THE DRAWINGS

FIG. 1 shows an example of a device architecture that supports electrical stimulation for haptic feedback in accordance with aspects of the present disclosure.

FIG. 2 shows an example of a process flow that supports electrical stimulation for haptic feedback in accordance with aspects of the present disclosure.

FIG. 3 illustrates an example of a system that supports electrical stimulation for haptic feedback in accordance with aspects of the present disclosure.

FIG. 4 illustrates an example of a system that supports electrical stimulation for haptic feedback in accordance with aspects of the present disclosure.

### DETAILED DESCRIPTION

Some wearable devices may be configured to provide feedback to users via alerts or other notifications, where the alerts or other notifications may communicate information about the health and well-being of the user or may communicate operating parameters of the wearable ring device, such as battery information (e.g., a low battery warning) or device settings (e.g., whether payment is enabled), among other information. Some wearable devices may be configured to emit lights, sounds, or vibrations to communicate the feedback to a user of the wearable device. However, such types of feedback may suffer from various inefficiencies or shortcomings. For example, some forms of feedback may disrupt an environment of the user, may be an annoyance to the user, may cause discomfort, or may fail to draw attention from the user, among other potential factors.

In accordance with examples described herein, a wearable ring device may be capable of providing electric-current based feedback as a form of feedback to a user. Electric-current based feedback may refer to an electric current generated by the wearable ring device that is felt as electrical stimulation by a finger of the user (e.g., electric current which stimulates nerves, muscles, or other tissues of the finger). Specifically, an inner housing of the wearable ring device may include an first electrode (e.g., an anode) and a second electrode (e.g., a cathode) such that when a user is wearing the wearable ring device, the wearable ring device may be capable of passing an electrical current from the first electrode to the second electrode (e.g., along an electrical path) through the finger of the user (e.g., external to the wearable ring device) that may be felt by the user as a form of electrical stimulation (e.g., electric-current based feedback). In some examples, respective positions of the first electrode and the second electrode may be relative to a nerve (e.g., or one or more nerves) in the finger of the user such that the electrical current may pass through the nerve (e.g., the electrical path may intersect the nerve). In such cases, electrical stimulation felt by the user may be felt along a length of the finger or in a fingertip, rather than at a location of the wearable ring device. In some other examples, the positions of the first electrode and the second electrode may not be nerve-dependent such that electrical stimulation felt by the user may be felt at the location of the wearable ring device.

In either case, the wearable ring device may deliver the electrical current (e.g., via the first electrode and the second electrode) as a sequence of pulses (e.g., pulse sequence) which are repeated at some frequency over a duration. By modulating a pulse sequence frequency, a pulse sequence duration, a stimulation current, or any combination thereof, the wearable ring device may be capable of generating multiple distinct sensations to convey different types of information. Additionally, or alternatively, the wearable ring device may adapt (e.g., adjust) a frequency (e.g., duration) of each pulse in the sequence of pulses on a pulse-by-pulse basis (e.g., may vary between each pulse, over a duration of a same feedback pattern). For example, the wearable ring device may modulate one or more first pulses in the sequence of pulses according to a first frequency (e.g., a pulse every 0.5 seconds) and may modulate one or more second pulses in the sequence of pulses according to a second frequency (e.g., a pulses every 1 second), where the second frequency is different than the first frequency. In other words, a duration of each pulse, a duration between each pulse, or both, in a sequence of pulses may vary (e.g., may not be the same). In some examples, these and other parameters may be modified according to one or more biomimetic stimulation pattern that may enable the wearable ring device to produce sensations (e.g., via the electrical stimulation) that may mimic (e.g., or almost mimic) natural sensations (e.g., pressure, touch) felt by the user (e.g., sensations generated by the human body) and that avoid sensations that may feel unnatural to the user or may cause discomfort. In some examples, the one or more biomimetic stimulation patterns may be based on physiological data collected by the wearable ring device, feedback from the user, or both.

In some examples, the wearable ring device may generate the electrical current based on a finger on which the user wears the wearable ring device. That is, a position of the nerve relative to the first electrode, the second electrode, or both, may vary between fingers. Thus, in some implementations, the wearable ring device may include multiple sets of electrodes (e.g., multiple anodes and multiple cathodes) and may obtain information (e.g., via user feedback, according to sensor information) that identifies which finger of the user the wearable ring device is being worn, such that the wearable ring device may select a set of electrodes (e.g., from the multiple sets of electrodes) that are capable of generating an electrical current along an electrical path that intersects a given nerve of the identified finger (e.g., targeting a specific nerve of the identified finger). Additionally, or alternatively, an intensity of electric stimulation, a duration of electric stimulation, or one or more other parameters associated with the electrical stimulation (e.g., associated with the sequence of pulses) may be configurable by the user according to a feedback mechanism. For example, the wearable ring device may generate one or more test instances of electrical stimulation, where each test instance is associated with a different set of parameters, and may prompt the user for feedback based on the one or more test instances. Thus, feedback provided from the user regarding the one or more test instances may enable the wearable ring device to adjust the electrical stimulation according to user preferences (e.g., according to the feedback). Additionally, or alternatively, after generating electrical stimulation via the first electrode and the second electrode to convey feedback to the user (e.g., during a non-test), the user may be capable of inputting feedback based on the electrical stimulation.

Aspects of the disclosure are initially described in the context of device architectures and process flows. Aspects of the disclosure are further described in the context of systems supporting physiological data collection from users via wearable devices.

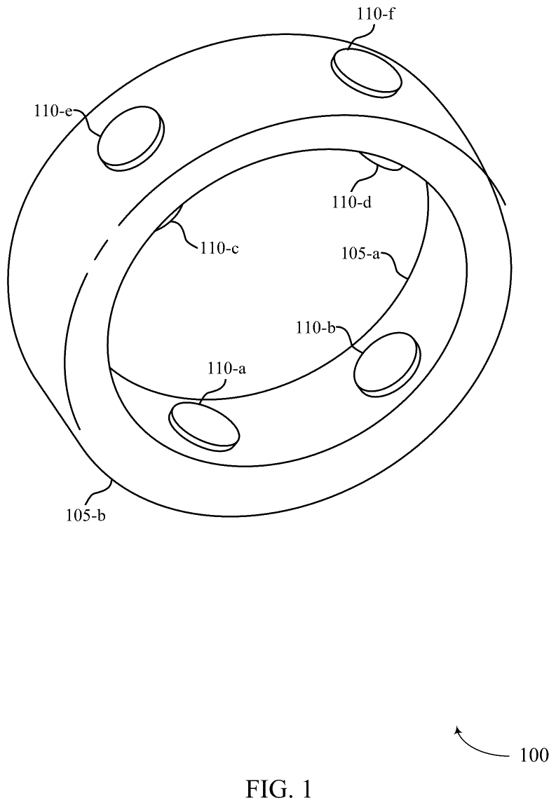

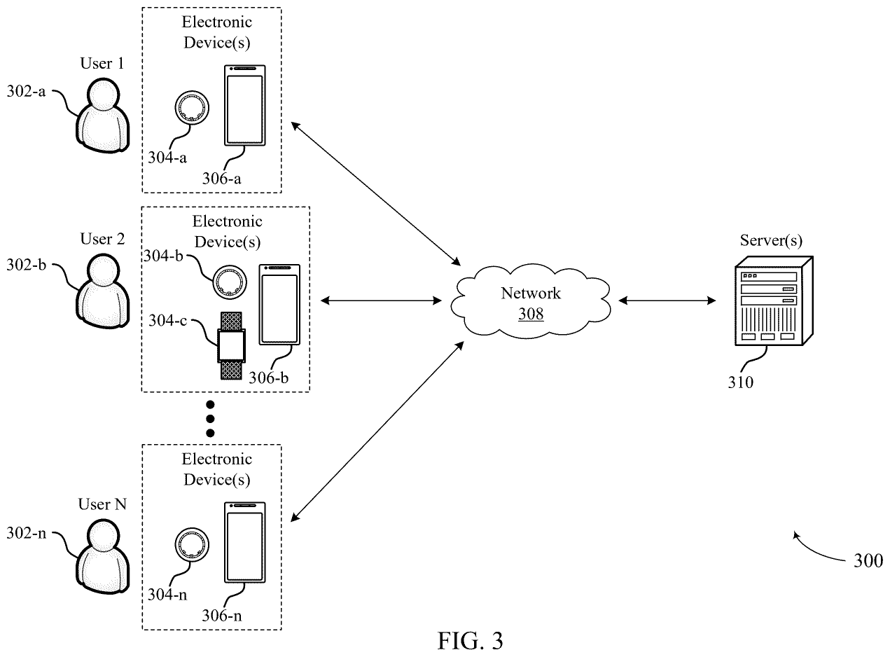

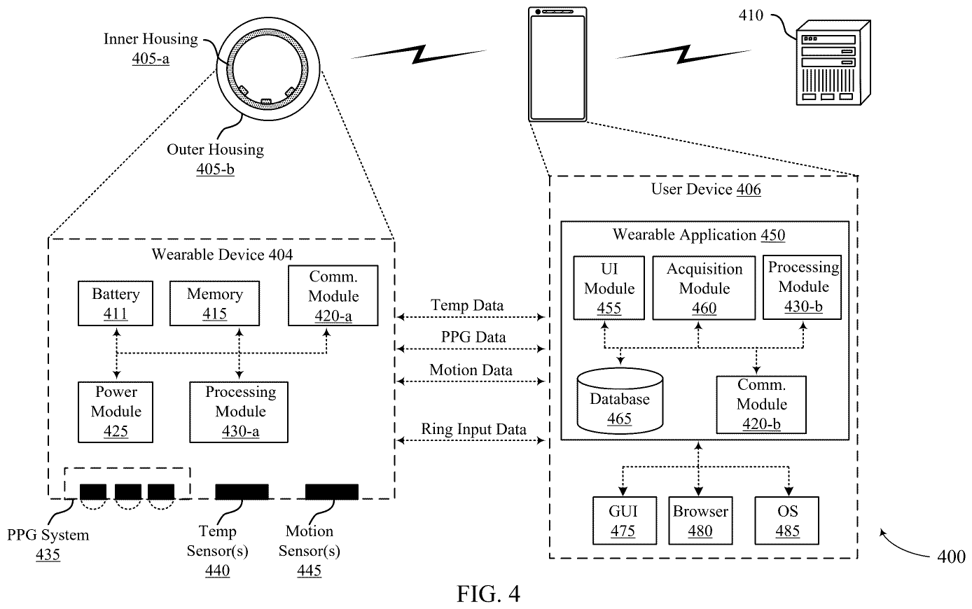

FIG. 1 shows an example of a wearable ring device 100 that supports electrical stimulation for haptic feedback in accordance with aspects of the present disclosure. The wearable ring device 100 may implement or may be implemented by aspects of a system 300, a system 400, or both, as described with reference to FIGS. 3 and 4. For example, the wearable ring device 100 may be an example of a wearable device 304, as described with reference to FIG. 3, or a wearable device 404, as described with reference to FIG. 4.

In some cases, the wearable ring device 100 may include multiple electrodes 110. For example, the wearable ring device 100 may include an electrode 110-a, an electrode 110-b, an electrode 110-c, and an electrode 110-d, an electrode 110-e an electrode 110-f, or any combination thereof. Though depicted and described in the context of six electrodes 110, this is not to be regarded as a limitation of the present disclosure. In this regard, the wearable ring device 100 may include any quantity of electrodes 110.

In such cases, one or more electrodes 110, such as the electrode 110-a, the electrode 110-b, the electrode 110-c, and the electrode 110-d, may be positioned relative to (e.g., at least partially within, on, embedded into, attached to) an inner surface 105-a (e.g., an inner housing) of the wearable ring device 100 and the one or more other electrodes 110, such as the electrode 110-e and the electrode 110-f, may be positioned relative to the outer surface 105-b (e.g., outer housing) of the wearable ring device 100. Additionally, or alternatively, at least a segment of the inner surface 105-a, the outer surface 105-b, or both, may be an electrode 110, may perform the functions of an electrode 110 as described herein, or both (e.g., rather than a separate component, at least a portion of the inner housing, the outer housing, or both, may function as an electrode 110). At least part of the outer surface 105-b may define an outer circumference of the wearable ring device 100 and at least part of the inner surface 105-a may define an inner circumference of the wearable ring device 100. In some cases, the electrode 110-a, the electrode 110-b, the electrode 110-c, and the electrode 110-d being positioned relative to the inner surface 105-a may result in the electrode 110-a, the electrode 110-b, the electrode 110-c, and the electrode 110-d contacting a finger of a user (e.g., a user 302, on a hand of the user 302) when the user is wearing the wearable ring device 100 (e.g., on the finger).

In some cases, the wearable ring device 100 may generate a current (e.g., electrical current) between a set of electrodes 110 (e.g., positioned relative to the inner surface 105-a) to provide electrical stimulation-based (e.g., electrical stimulation based) haptic feedback to the user. For example, the wearable ring device 100 may generate the current from (e.g., through) the electrode 110-a to (e.g., through) the electrode 110-b along an electrical path that is external to the wearable ring device 100 and that passes through human tissue of the finger of the user. In some cases, the electrical path may travel (e.g., may be) within a threshold proximity of (e.g., through) a nerve of the finger on which the wearable ring device 100 is worn (e.g., may intersect the nerve). In such cases, the current generated between the set of electrodes 110 may induce (e.g., produce, result in) electrical stimulation (e.g., one or more tactile sensations) at another location on the finger of the user (e.g., within a threshold proximity of the wearable ring device 100), different from a location of the wearable ring device 100 (e.g., the nerve of the user may detect the electrical stimulation). For example, the user may feel the electrical stimulation along a length of the finger or in a fingertip, rather than at a location of the wearable ring device 100. Additionally, or alternatively, the electrical path may travel outside of the threshold proximity of the nerve of the finger on which the wearable ring device 100 is worn (e.g., may not intersect the nerve, skin of the user may detect the electrical stimulation). In such cases, the current generated between the set of electrodes 110 may induce a sensation (e.g., via electrical stimulation) at the location of the wearable ring device 100.

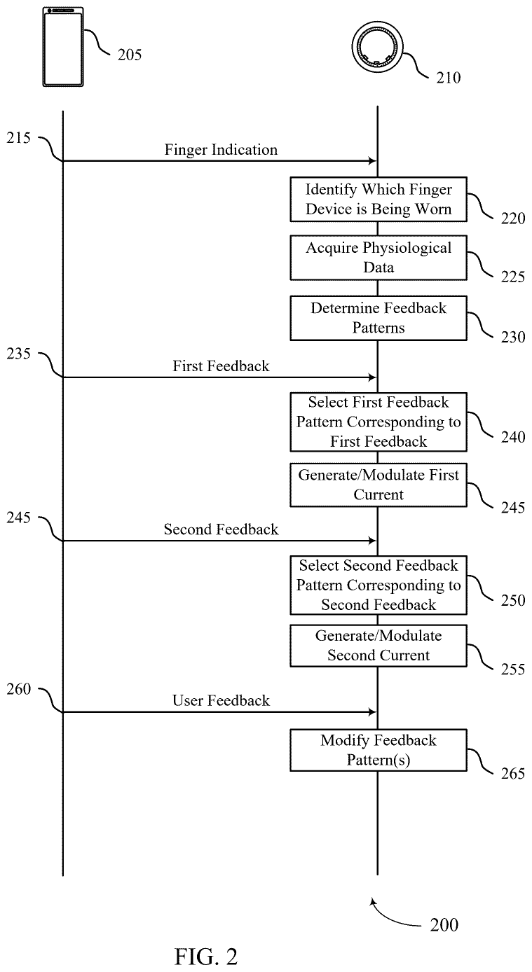

In some cases, generation of a current along an electrical path may be based on selecting which set of electrodes 110 of the multiple electrodes 110 are used to generate the current. In a first example, the wearable ring device 100 may generate a first current along a first electrical path that is within the threshold proximity of a first nerve (e.g., an ulnar nerve of a fourth or fifth finger of the user). In such cases, the wearable ring device 100 may select the electrode 110-a and the electrode 110-b for generation of the first current along the first electrical path. In a second example, the wearable ring device 100 may generate a second current along a second electrical path that is within the threshold proximity of a second nerve (e.g., a median nerve of a first, second, or third finger of the user). In such cases, the wearable ring device 100 may select the electrode 110-c and the electrode 110-d for generation of the second current. In some examples, which nerve to target with the electric current may be based on which finger the wearable ring device 100 is being worn by the user, which is described in greater detail with reference to FIG. 2.

In some cases, the wearable ring device 100 may be capable of generating multiple feedback patterns (e.g., electric current-based feedback patterns). In some examples, the multiple feedback patterns may be based on which set of electrodes 110 are used to generate the current. For example, as described previously, in some cases, the current generated between the set of electrodes 110 may induce electrical stimulation along the length of the finger or in the fingertip, rather than at the location of the wearable ring device 100, based on a corresponding electrical path being within the threshold proximity of a nerve of the user and, in some other cases, the current generated between the set of electrodes 110 may induce electrical stimulation at the location of the wearable ring device 100 based on a corresponding electrical path being outside of the threshold proximity of the nerve of the user. As such, a first feedback pattern may be associated with electrical stimulation along the length of the finger or in the fingertip and a second feedback pattern may be associated with electrical stimulation at the location of the wearable ring device 100.

Additionally, or alternatively, the multiple feedback patterns may be associated with one or more electrical parameters associated with generation of the current. For example, the wearable ring device 100 may be capable of modulating a frequency of the current between the set of electrodes 110 according to a selected feedback pattern. Other electrical parameters may include, but may not be limited to, a frequency of the current, a modulation duration, a modulation amplitude, a modulation pattern, a voltage of the current (e.g., intensity of electrical stimulation), or any combination thereof. Thus, the wearable ring device may modify, or adjust, the one or more electrical parameters of the current may be modified according to a selected feedback pattern. For example, a current (e.g., sequence of pulses) generated between the set of electrodes 110 may be a sequence of 250 microsecond (μs) square pulses (e.g., biphasic pulses), may have a current of 2-10 milliampere (mA), may have a frequency of 10-100 Hertz (Hz) (e.g., biphasic pulses are repeated with a carrier frequency), and may have a charge of 1-3 microcoulombs (μC) per pulse in the sequence of pulses.

Additionally, or alternatively, the multiple feedback patterns may be based on physiological data (e.g., bioimpedance data) associated with the user (e.g., measured by the wearable ring device), feedback from the user, or both. For example, the one or more electrical parameters may be selected by the wearable ring device 100 to produce biomimetic stimulation (e.g., biomimetic electrical stimulation). In other words, the electrical stimulation produced by the current (e.g., in accordance with the one or more electrical parameters) may mimic biometric sensations naturally produced by the user (e.g., present in a body of the user). As such, to mimic the natural biometric stimulation, the wearable ring device may select the one or more electric parameters based on the physiological data associated with the user, feedback from the user indicative of how the electrical stimulation feels, or both. In some cases, one or more feedback patterns that are biomimetic may be referred to as one or more biomimetic stimulation patterns, which may produce sensations that mimic natural pressure and touch (e.g., that mimic sensor feedback used in intraneural sensory feedback or asynchronous stochastic neural activity).

In some examples, a position of each electrode 110 in the set of electrodes 110 relative to the inner surface 105-a (e.g., and relative to each other) may be based on a size of the wearable ring device 100. For example, a first size (e.g., a larger size) of the wearable ring device 100 may result in the set of electrodes 110 being spaced apart by a first radial distance (e.g., a larger distance), and a second size (e.g., a smaller size) of the wearable ring device 100 may result in the set of electrodes 110 being space spaced apart by a second radial distance (e.g., a smaller), where the first distance is larger than the second distance based on the first size being larger than the second size. Thus, in some examples, a voltage of the current may be based on the size of the wearable ring device 100. That is, because the set of electrodes 110 are further apart from each other in the first size than the second size, the wearable ring device 100 may generate the current at a higher voltage in the first size (e.g., than the second ring size) to produce a same electrical stimulation (e.g., sensation) as the second size.

In some cases, the wearable ring device 100 may measure (e.g., collect) physiological data associated with the user, such as electrocardiogram (ECG) (e.g., or EKG) data, bioimpedance (BioZ) data, electrodermal (EDA) data, or any combination thereof, when the user contacts (e.g., touches) at least one of the electrode 110-e and the electrode 110-f). In such cases, the user may contact at least one of the electrode 110-e and the electrode 110-f with another hand of the user (e.g., a second hand of the user), a second finger on the other hand of the user, an opposite wrist of the user (e.g., an opposite wrist than a wrist associated with the first hand), or the like thereof. When the user contacts at least one of the electrode 110-a and the electrode 110-b, one or more electrical paths (e.g., signal paths) may be created (e.g., may be available, may be present) between any combination of the electrode 110-e (e.g., if contacted), the electrode 110-f (e.g., if contacted), the electrode 110-a, the electrode 110-b, the electrode 110-c, and the electrode 110-d, (e.g., through a body of the user), where the physiological data is based on the one or more signal paths (e.g., collected via transmission of one or more electrical signals via the one or more signal paths). For example, the wearable ring device 100 may measure EDA data based on the user contacting at least two of the electrodes 110, may measure ECG data based on the user contacting at least three of the electrodes 110, and may measure BioZ data based on the user contacting at least four of the electrodes 110.

Due to the dual capability of the electrodes 110, in some cases, the wearable ring device 100 may switch between measurement of physiological data via at least a subset of the electrodes 110 and generation of the current between the set of electrodes 110 for electric current-based haptic feedback. In other words, at a first time, the wearable ring device may measure physiological data via the at least subset of the electrodes and, at a second time, may generate a current between the set of electrodes for electric current-based haptic feedback. For example, at the first time, the wearable ring device 100 may measure ECG data based on the user contacting the electrode 110-a, the electrode 110-b, the electrode 110-e, and the electrode 110-f and, at the second time, later than the first time, may generate a current between the electrode 110-a and the electrode 110-b for electric current-based haptic feedback.

FIG. 2 shows an example of a process flow 200 that supports electrical stimulation for haptic feedback in accordance with aspects of the present disclosure. Aspects of the process flow 200 may be performed by a user device 205 and a wearable device 210. The user device 205 may have a wireless connection with the wearable device 210 such that wireless communications (e.g., over-the-air, electromagnetic signals) can be exchanged between the user device 205 and the wearable device 210. The wearable device 210 may be an example of a wearable device 404, as described with reference to FIG. 4.

At 215, the wearable device 210 may receive (e.g., from the user device 205) an indication of which finger of a user the wearable device 210 is being worn. Additionally, or alternatively, at 220, the wearable device 210 may identify which finger of the user the wearable device 210 is being worn. In some cases, the wearable device 210 may autonomously identify (e.g., without feedback from the user device 205) the finger of the user based on data from one or more sensors of the wearable ring device.

At 225, the wearable device 210 may acquire physiological data of the user via the one or more sensors of the wearable device 210. Additionally, or alternatively, the wearable device 210 may acquire the physiological data based on generating a current along an electrical path between a first electrode positioned on an inner housing of the wearable device 210 and a third electrode positioned on an outer housing of the wearable device 210 (e.g., through a body of the user), as described in greater detail with reference to FIG. 1. In some examples, generating the current to acquire the physiological data and generating a second current to provide electric current-based feedback may occur at different times. In some examples, the physiological data may be ECG (e.g., or EKG) data, BioZ data, EDA data, or any combination thereof.

At 230, the wearable device 210 may determine a set of feedback patterns configured for output of electric current-based feedback to the user. The wearable device 210 may determine the set of feedback patterns based on the identification of which finger the wearable device is worn, the acquired physiological data of the user, or both.

In some cases, the set of feedback patterns may be based on different sets of electrodes (e.g., a first electrode and a second electrode) which enable a generation of current along varying (e.g., multiple) electrical paths external to the wearable device 210. In some examples, a first electrical path between a first set of electrodes may intersect (e.g., pass within a threshold proximity of) a first nerve of a first finger of the user. A second electrical path between a second set of electrodes may intersect a second nerve of a second finger of the user, which may be of a same finger or a different finger than the first finger. In some examples, the wearable device 210 may determine the set of feedback patterns based on identification of which finger the wearable device 210 is being worn. That is, the wearable device may select feedback patterns (e.g., for inclusion in a set of enabled feedback patterns) which may be configured for the identified finger (e.g., or identified nerve) and may exclude feedback patterns which are configured for non-identified fingers.

In some cases, the set of feedback patterns may differ with respect to one or more electrical parameters. For example, each feedback pattern of the set of feedback patterns may be associated with a respective frequency of the current, a respective modulation duration, a respective modulation amplitude, a respective modulation pattern, a respective voltage of the current, or a combination thereof.

In some examples, varying feedback patterns may be configured for different types of feedback to be communicated. For example, generation of a current in accordance with a first feedback pattern may communicate to the user that a battery level of the wearable device 210 is low, whereas generation of the current in accordance with a second feedback pattern may communicate to the user that payment is enabled. Additionally, or alternatively, the different types of feedback may include feedback relating to the acquired physiological data (e.g., that the user has been idle for a threshold amount of time, that the user may be drowsy).

At 235, the wearable device 210 may identify (e.g., receive from the user device 205, determine at the wearable device 210) first feedback to be communicated to the user. In some cases, the first feedback may alert the user of a physiological state of the user, may communicate one or more system alerts (e.g., that payment has been enabled, that battery level is low), or any other potential feedback to be communicated.

At 240, the wearable device 210 may select (e.g., from a set of candidate feedback patterns) a first feedback pattern that corresponds to the first feedback. Selecting the first feedback pattern may include selecting a first set of electrodes for generation of the current, selecting one or more first electrical parameters, or both.

At 245, the wearable device 210 may generate a first current between a first electrode and a second electrode (e.g., according to the first set of electrodes 110) via a first electrical path external to the wearable device 210. In such cases, the wearable device 210 may generate the first current according to the one or more first electrical parameters that correspond to the first feedback pattern. For example, the wearable device 210 may generate (e.g., and modulate) the first current according to a first frequency, a first modulation duration, a first modulation amplitude, a first modulation pattern, a first voltage, or a combination thereof.

At 245, the wearable device 210 may identify (e.g., receive from the user device 205, determine at the wearable device 210) second feedback to be communicated to the user.

At 250, the wearable device 210 may select (e.g., from the set of candidate feedback patterns) a second feedback pattern that corresponds to the second feedback. Selecting the second feedback pattern may include selecting a second set of electrodes for generation of the current, selecting one or more second electrical parameters, or both.

At 255, the wearable device 210 may generate a second current between a third electrode and a fourth electrode (e.g., according to the second set of electrodes 110) via a second electrical path external to the wearable device 210. The third and fourth electrodes may be the same as or different from the first and second electrodes. For example, one or both electrodes may be reused for generation of the second current, or different electrodes may be used.

Additionally, or alternatively, the wearable device 210 may generate the second current according to the one or more second electrical parameters that correspond to the selected second feedback pattern. For example, the wearable device 210 may generate (e.g., and modulate) the second current according to a second frequency, a second modulation duration, a second modulation amplitude, a second modulation pattern, a second voltage, or a combination thereof. In some examples, at least a subset of the one or more second electrical parameters may be the same as or different than the one or more first electrical parameters.

At 260, the wearable device 210 may receive user feedback (e.g., from the user device 205) based on generating the first current according to the first feedback pattern, the second current according to the second feedback pattern, or both. In some cases, the wearable device 210 may enter a feedback configuration mode. In the feedback configuration mode, the wearable device 210 may output one or more test (e.g., default) currents associated with one or more default electrical parameters and may receive feedback from the user in response to the test currents. In some examples, the feedback from the user may be based on one or more user inputs at the user device 205, the wearable device 210, or both. Additionally, or alternatively, the feedback from the user may be based on physiological data associated with the user (e.g., collected from one or more sensors) in response to the generated first current or second current.

At 265, the wearable device 210 may modify at least a subset of the one or more first electrical parameters, the one or more second electrical parameters, or both, based on the received user feedback. Additionally, or alternatively, the wearable device 210 may modify which set of electrodes are configured to generate the first feedback pattern or the second feedback pattern based on the received user feedback. Accordingly, the wearable device 210 may generate future instances of the first feedback pattern or the second feedback pattern according to the modifications.

FIG. 3 illustrates an example of a system 300 that supports electrical stimulation for haptic feedback in accordance with aspects of the present disclosure. The system 300 includes a plurality of electronic devices (e.g., wearable devices 304, user devices 306) that may be worn and/or operated by one or more users 302. The system 300 further includes a network 308 and one or more servers 310.

The electronic devices may include any electronic devices known in the art, including wearable devices 304 (e.g., ring wearable devices, watch wearable devices, etc.), user devices 306 (e.g., smartphones, laptops, tablets). The electronic devices associated with the respective users 302 may include one or more of the following functionalities: 1) measuring physiological data, 2) storing the measured data, 3) processing the data, 4) providing outputs (e.g., via GUIs) to a user 302 based on the processed data, and 5) communicating data with one another and/or other computing devices. Different electronic devices may perform one or more of the functionalities.

Example wearable devices 304 may include wearable computing devices, such as a ring computing device (hereinafter “ring”) configured to be worn on a user's 302 finger, a wrist computing device (e.g., a smart watch, fitness band, or bracelet) configured to be worn on a user's 302 wrist, and/or a head mounted computing device (e.g., glasses/goggles). Wearable devices 304 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 304 may also be attached to, or included in, articles of clothing. For example, wearable devices 304 may be included in pockets and/or pouches on clothing. As another example, wearable device 304 may be clipped and/or pinned to clothing, or may otherwise be maintained within the vicinity of the user 302. Example articles of clothing may include, but are not limited to, hats, shirts, gloves, pants, socks, outerwear (e.g., jackets), and undergarments. In some implementations, wearable devices 304 may be included with other types of devices such as training/sporting devices that are used during physical activity. For example, wearable devices 304 may be attached to, or included in, a bicycle, skis, a tennis racket, a golf club, and/or training weights.

Much of the present disclosure may be described in the context of a wearable device 304, which may include finger-worn wearable devices, wrist-worn wearable devices, and the like. Accordingly, the terms “wearable device 304,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 306 may include handheld mobile computing devices, such as smartphones and tablet computing devices. User devices 306 may also include personal computers, such as laptop and desktop computing devices. Other example user devices 306 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 306 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 304, user devices 306) may measure physiological parameters of respective users 302, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 304), mobile device application, or a server computing device may process received physiological data that was measured by other devices.

In some implementations, a user 302 may operate, or may be associated with, multiple electronic devices, some of which may measure physiological parameters and some of which may process the measured physiological parameters. In some implementations, a user 302 may have a ring (e.g., wearable device 304) that measures physiological parameters. The user 302 may also have, or be associated with, a user device 306 (e.g., mobile device, smartphone), where the wearable device 304 and the user device 306 are communicatively coupled to one another. In some cases, the user device 306 may receive data from the wearable device 304 and perform some/all of the calculations described herein. In some implementations, the user device 306 may also measure physiological parameters described herein, such as motion/activity parameters.

For example, as illustrated in FIG. 3, a first user 302-a (User 1) may operate, or may be associated with, a wearable device 304-a (e.g., wearable ring device) and a user device 306-a that may operate as described herein. In this example, the user device 306-a associated with user 302-a may process/store physiological parameters measured by the wearable device 304-a. Comparatively, a second user 302-b (User 2) may be associated with a wearable device 304-b (e.g., wearable ring device), a wearable device 304-c (e.g., a wrist-worn wearable device, such as a watch), and a user device 306-b, where the user device 306-b associated with user 302-b may process/store physiological parameters measured by the wearable device 304-b and 304-c. Moreover, an nth user 302-n (User N) may be associated with an arrangement of electronic devices described herein (e.g., wearable device 304-n, user device 306-n). In some aspects, wearable devices 304 (e.g., wearable ring devices, wrist-worn wearable devices) and other electronic devices may be communicatively coupled to the user devices 306 of the respective users 302 via Bluetooth, Wi-Fi, and other wireless protocols. Moreover, in some cases, the wearable device 304 and the user device 306 may be included within (or make up) the same device. For example, in some cases, the wearable device 304 may be configured to execute an application associated with the wearable device 304, and may be configured to display data via a GUI.

In some implementations, the wearable devices 304 (e.g., wearable ring devices) of the system 300 may be configured to collect physiological data from the respective users 302 based on arterial blood flow within the user's finger. In particular, a wearable device 304 (e.g., wearable ring device) may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 300 may be configured to collect physiological data from the respective users 302 based on blood flow diffused into a microvascular bed of skin with capillaries and arterioles. For example, the system 300 may collect PPG data based on a measured amount of blood diffused into the microvascular system of capillaries and arterioles. In some implementations, the wearable device 304 (e.g., wearable ring device) may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable ring device has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 300 (e.g., user devices 306, wearable devices 304) may be communicatively coupled to one or more servers 310 via wired or wireless communication protocols. For example, as shown in FIG. 3, the electronic devices (e.g., user devices 306) may be communicatively coupled to one or more servers 310 via a network 308. The network 308 may implement transfer control protocol and internet protocol (TCP/IP), such as the Internet, or may implement other network 308 protocols. Network connections between the network 308 and the respective electronic devices may facilitate transport of data via email, web, text messages, mail, or any other appropriate form of interaction within a computer network 308. For example, in some implementations, the wearable device 304-a associated with the first user 302-a may be communicatively coupled to the user device 306-a, where the user device 306-a is communicatively coupled to the servers 310 via the network 308. In additional or alternative cases, wearable devices 304 (e.g., wearable ring devices, wrist-worn wearable devices such as watches) may be directly communicatively coupled to the network 308.

The system 300 may offer an on-demand database service between the user devices 306 and the one or more servers 310. In some cases, the servers 310 may receive data from the user devices 306 via the network 308, and may store and analyze the data. Similarly, the servers 310 may provide data to the user devices 306 via the network 308. In some cases, the servers 310 may be located at one or more data centers. The servers 310 may be used for data storage, management, and processing. In some implementations, the servers 310 may provide a web-based interface to the user device 306 via web browsers.

In some aspects, the system 300 may detect periods of time that a user 302 is asleep, and classify periods of time that the user 302 is asleep into one or more sleep stages (e.g., sleep stage classification). For example, as shown in FIG. 3, User 302-a may be associated with a wearable device 304-a (e.g., a wearable ring device) and a user device 306-a. In this example, the wearable device 304-a may collect physiological data associated with the user 302-a, including temperature, heart rate, HRV, respiratory rate, and the like. In some aspects, data collected by the wearable device 304-a may be input to a machine learning classifier, where the machine learning classifier is configured to determine periods of time that the user 302-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 302-a via a GUI of the user device 306-a. Sleep stage classification may be used to provide feedback to a user 302-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 300 may utilize circadian rhythm-derived features to further improve physiological data collection, data processing procedures, and other techniques described herein. The term circadian rhythm may refer to a natural, internal process that regulates an individual's sleep-wake cycle that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 302-a via the wearable device 304-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 302 to generate tailored, individualized circadian rhythm adjustment models that are specific to each respective user 302.

In some aspects, the system 300 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 300 may support techniques for generation of electric current-based feedback, which may be provided to a user 302 of a wearable device 304 through generation of an electric current between a set of electrodes of the wearable device 304. The wearable device 304 may generate electric current according to a sequence of pulses which correspond to a type of feedback to be provided to the user 302.

It should be appreciated by a person skilled in the art that one or more aspects of the disclosure may be implemented in a system 300 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

FIG. 4 illustrates an example of a system 400 that supports electrical stimulation for haptic feedback in accordance with aspects of the present disclosure. The system 400 may implement, or be implemented by, system 300. In particular, system 400 illustrates an example of a wearable device 404 (e.g., wearable ring device), a user device 406, and a server 410, as described with reference to FIG. 3.

In some aspects, the wearable device 404 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 400 further includes a user device 406 (e.g., a smartphone) in communication with the wearable device 404. For example, the wearable device 404 may be in wireless and/or wired communication with the user device 406. In some implementations, the wearable device 404 may send measured and processed data (e.g., temperature data, photoplethysmogram (PPG) data, motion/accelerometer data, ring input data, and the like) to the user device 406. The user device 406 may also send data to the wearable device 404, such as wearable device 404 firmware/configuration updates. The user device 406 may process data. In some implementations, the user device 406 may transmit data to the server 410 for processing and/or storage.

The wearable device 404 may include a housing 405 that may include an inner housing 405-a and an outer housing 405-b. In some aspects, the inner housing 405-a, the outer housing 405-b, or both, may include a curved profile/surface. In particular, the housing 405 may exhibit any curved or “circumferential” profile, including a circular profile, an elliptical profile, and the like. Moreover, in some cases, the inner housing 405-a, the outer housing 405-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 405 of the wearable device 404 may store or otherwise include various components of the ring including, but not limited to, device electronics, a power source (e.g., battery 411, and/or capacitor), one or more substrates (e.g., printable circuit boards) that interconnect the device electronics and/or power source, and the like. The device electronics may include device modules (e.g., hardware/software), such as: a processing module 430-a, a memory 415, a communication module 420-a, a power module 425, and the like. The device electronics may also include one or more sensors. Example sensors may include one or more temperature sensors 440, a PPG sensor assembly (e.g., PPG system 435), and one or more motion sensors 445.

The sensors may include associated modules (not illustrated) configured to communicate with the respective components/modules of the wearable device 404, and generate signals associated with the respective sensors. In some aspects, each of the components/modules of the wearable device 404 may be communicatively coupled to one another via wired or wireless connections. Moreover, the wearable device 404 may include additional and/or alternative sensors or other components that are configured to collect physiological data from the user, including light sensors (e.g., LEDs), oximeters, and the like.

The wearable device 404 shown and described with reference to FIG. 4 is provided solely for illustrative purposes. As such, the wearable device 404 may include additional or alternative components as those illustrated in FIG. 4. Other wearable devices 404 that provide functionality described herein may be fabricated. For example, wearable devices 404 with fewer components (e.g., sensors) may be fabricated. In a specific example, a wearable device 404 with a single temperature sensor 440 (or other sensor), a power source, and device electronics configured to read the single temperature sensor 440 (or other sensor) may be fabricated. In another specific example, a temperature sensor 440 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 440 (or other sensor). In other examples, a wearable device 404 that includes additional sensors and processing functionality may be fabricated.

The housing 405 may include one or more housing 405 components. The housing 405 may include an outer housing 405-b component (e.g., a shell) and an inner housing 405-a component (e.g., a molding). The housing 405 may include additional components (e.g., additional layers) not explicitly illustrated in FIG. 4. For example, in some implementations, the wearable device 404 may include one or more insulating layers that electrically insulate the device electronics and other conductive materials (e.g., electrical traces) from the outer housing 405-b (e.g., a metal outer housing 405-b). The housing 405 may provide structural support for the device electronics, battery 411, substrate(s), and other components. For example, the housing 405 may protect the device electronics, battery 411, and substrate(s) from mechanical forces, such as pressure and impacts. The housing 405 may also protect the device electronics, battery 411, and substrate(s) from water and/or other chemicals.

The outer housing 405-b may be fabricated from one or more materials. In some implementations, the outer housing 405-b may include a metal, such as titanium, that may provide strength and abrasion resistance at a relatively light weight. The outer housing 405-b may also be fabricated from other materials, such polymers. In some implementations, the outer housing 405-b may be protective as well as decorative.

The inner housing 405-a may be configured to interface with the user's finger. The inner housing 405-a may be formed from a polymer (e.g., a medical grade polymer) or other material. In some implementations, the inner housing 405-a may be transparent. For example, the inner housing 405-a may be transparent to light emitted by the PPG light emitting diodes (LEDs). In some implementations, the inner housing 405-a component may be molded onto the outer housing 405-b. For example, the inner housing 405-a may include a polymer that is molded (e.g., injection molded) to fit into an outer housing 405-b metallic shell.

The wearable device 404 may include one or more substrates (not illustrated). The device electronics and battery 411 may be included on the one or more substrates. For example, the device electronics and battery 411 may be mounted on one or more substrates. Example substrates may include one or more printed circuit boards (PCBs), such as flexible PCB (e.g., polyimide). In some implementations, the electronics/battery 411 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 411 to the device electronics.

The device electronics, battery 411, and substrates may be arranged in the wearable device 404 in a variety of ways. In some implementations, one substrate that includes device electronics may be mounted along the bottom of the wearable device 404 (e.g., the bottom half), such that the sensors (e.g., PPG system 435, temperature sensors 440, motion sensors 445, and other sensors) interface with the underside of the user's finger. In these implementations, the battery 411 may be included along the top portion of the wearable device 404 (e.g., on another substrate).

The various components/modules of the wearable device 404 represent functionality (e.g., circuits and other components) that may be included in the wearable device 404. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 415 (memory module) of the wearable device 404 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 415 may store any of the data described herein. For example, the memory 415 may be configured to store data (e.g., motion data, temperature data, PPG data) collected by the respective sensors and PPG system 435. Furthermore, memory 415 may include instructions that, when executed by one or more processing circuits, cause the modules to perform various functions attributed to the modules herein. The device electronics of the wearable device 404 described herein are only example device electronics. As such, the types of electronic components used to implement the device electronics may vary based on design considerations.

The functions attributed to the modules of the wearable device 404 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 430-a of the wearable device 404 may include one or more processors (e.g., processing units), microcontrollers, digital signal processors, systems on a chip (SOCs), and/or other processing devices. The processing module 430-a communicates with the modules included in the wearable device 404. For example, the processing module 430-a may transmit/receive data to/from the modules and other components of the wearable device 404, such as the sensors. As described herein, the modules may be implemented by various circuit components. Accordingly, the modules may also be referred to as circuits (e.g., a communication circuit and power circuit).

The processing module 430-a may communicate with the memory 415. The memory 415 may include computer-readable instructions that, when executed by the processing module 430-a, cause the processing module 430-a to perform the various functions attributed to the processing module 430-a herein. In some implementations, the processing module 430-a (e.g., a microcontroller) may include additional features associated with other modules, such as communication functionality provided by the communication module 420-a (e.g., an integrated Bluetooth Low Energy transceiver) and/or additional onboard memory 415.

The communication module 420-a may include circuits that provide wireless and/or wired communication with the user device 406 (e.g., communication module 420-b of the user device 406). In some implementations, the communication modules 420-a, 420-b may include wireless communication circuits, such as Bluetooth circuits and/or Wi-Fi circuits. In some implementations, the communication modules 420-a, 420-b can include wired communication circuits, such as Universal Serial Bus (USB) communication circuits. Using the communication module 420-a, the wearable device 404 and the user device 406 may be configured to communicate with each other. The processing module 430-a of the ring may be configured to transmit/receive data to/from the user device 406 via the communication module 420-a. Example data may include, but is not limited to, motion data, temperature data, pulse waveforms, heart rate data, HRV data, PPG data, and status updates (e.g., charging status, battery charge level, and/or wearable device 404 configuration settings). The processing module 430-a of the ring may also be configured to receive updates (e.g., software/firmware updates) and data from the user device 406.

The wearable device 404 may include a battery 411 (e.g., a rechargeable battery 411). An example battery 411 may include a Lithium-Ion or Lithium-Polymer type battery 411, although a variety of battery 411 options are possible. The battery 411 may be wirelessly charged. In some implementations, the wearable device 404 may include a power source other than the battery 411, such as a capacitor. The power source (e.g., battery 411 or capacitor) may have a curved geometry that matches the curve of the wearable device 404. In some aspects, a charger or other power source may include additional sensors that may be used to collect data in addition to, or that supplements, data collected by the wearable device 404 itself. Moreover, a charger or other power source for the wearable device 404 may function as a user device 406, in which case the charger or other power source for the wearable device 404 may be configured to receive data from the wearable device 404, store and/or process data received from the wearable device 404, and communicate data between the wearable device 404 and the servers 410.

In some aspects, the wearable device 404 includes a power module 425 that may control charging of the battery 411. For example, the power module 425 may interface with an external wireless charger that charges the battery 411 when interfaced with the wearable device 404. The charger may include a datum structure that mates with a wearable device 404 datum structure to create a specified orientation with the wearable device 404 during charging. The power module 425 may also regulate voltage(s) of the device electronics, regulate power output to the device electronics, and monitor the state of charge of the battery 411. In some implementations, the battery 411 may include a protection circuit module (PCM) that protects the battery 411 from high current discharge, over voltage during charging, and under voltage during discharge. The power module 425 may also include electro-static discharge (ESD) protection.

The one or more temperature sensors 440 may be electrically coupled to the processing module 430-a. The temperature sensor 440 may be configured to generate a temperature signal (e.g., temperature data) that indicates a temperature read or sensed by the temperature sensor 440. The processing module 430-a may determine a temperature of the user in the location of the temperature sensor 440. For example, in the wearable device 404, temperature data generated by the temperature sensor 440 may indicate a temperature of a user at the user's finger (e.g., skin temperature). In some implementations, the temperature sensor 440 may contact the user's skin. In other implementations, a portion of the housing 405 (e.g., the inner housing 405-a) may form a barrier (e.g., a thin, thermally conductive barrier) between the temperature sensor 440 and the user's skin. In some implementations, portions of the wearable device 404 configured to contact the user's finger may have thermally conductive portions and thermally insulative portions. The thermally conductive portions may conduct heat from the user's finger to the temperature sensors 440. The thermally insulative portions may insulate portions of the wearable device 404 (e.g., the temperature sensor 440) from ambient temperature.

In some implementations, the temperature sensor 440 may generate a digital signal (e.g., temperature data) that the processing module 430-a may use to determine the temperature. As another example, in cases where the temperature sensor 440 includes a passive sensor, the processing module 430-a (or a temperature sensor 440 module) may measure a current/voltage generated by the temperature sensor 440 and determine the temperature based on the measured current/voltage. Example temperature sensors 440 may include a thermistor, such as a negative temperature coefficient (NTC) thermistor, or other types of sensors including resistors, transistors, diodes, and/or other electrical/electronic components.

The processing module 430-a may sample the user's temperature over time. For example, the processing module 430-a may sample the user's temperature according to a sampling rate. An example sampling rate may include one sample per second, although the processing module 430-a may be configured to sample the temperature signal at other sampling rates that are higher or lower than one sample per second. In some implementations, the processing module 430-a may sample the user's temperature continuously throughout the day and night. Sampling at a sufficient rate (e.g., one sample per second) throughout the day may provide sufficient temperature data for analysis described herein.

The processing module 430-a may store the sampled temperature data in memory 415. In some implementations, the processing module 430-a may process the sampled temperature data. For example, the processing module 430-a may determine average temperature values over a period of time. In one example, the processing module 430-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 415 may store the average temperature values over time. In some implementations, the memory 415 may store average temperatures (e.g., one per minute) instead of sampled temperatures in order to conserve memory 415.

The sampling rate, which may be stored in memory 415, may be configurable. In some implementations, the sampling rate may be the same throughout the day and night. In other implementations, the sampling rate may be changed throughout the day/night. In some implementations, the wearable device 404 may filter/reject temperature readings, such as large spikes in temperature that are not indicative of physiological changes (e.g., a temperature spike from a hot shower). In some implementations, the wearable device 404 may filter/reject temperature readings that may not be reliable due to other factors, such as excessive motion during exercise (e.g., as indicated by a motion sensor 445).

The wearable device 404 (e.g., communication module) may transmit the sampled and/or average temperature data to the user device 406 for storage and/or further processing. The user device 406 may transfer the sampled and/or average temperature data to the server 410 for storage and/or further processing.

Although the wearable device 404 is illustrated as including a single temperature sensor 440, the wearable device 404 may include multiple temperature sensors 440 in one or more locations, such as arranged along the inner housing 405-a near the user's finger. In some implementations, the temperature sensors 440 may be stand-alone temperature sensors 440. Additionally, or alternatively, one or more temperature sensors 440 may be included with other components (e.g., packaged with other components), such as with the accelerometer and/or processor.

The processing module 430-a may acquire and process data from multiple temperature sensors 440 in a similar manner described with respect to a single temperature sensor 440. For example, the processing module 430 may individually sample, average, and store temperature data from each of the multiple temperature sensors 440. In other examples, the processing module 430-a may sample the sensors at different rates and average/store different values for the different sensors. In some implementations, the processing module 430-a may be configured to determine a single temperature based on the average of two or more temperatures determined by two or more temperature sensors 440 in different locations on the finger.

The temperature sensors 440 on the wearable device 404 may acquire distal temperatures at the user's finger (e.g., any finger). For example, one or more temperature sensors 440 on the wearable device 404 may acquire a user's temperature from the underside of a finger or at a different location on the finger. In some implementations, the wearable device 404 may continuously acquire distal temperature (e.g., at a sampling rate). Although distal temperature measured by a wearable device 404 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the wearable device 404 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 404 may include a PPG system 435. The PPG system 435 may include one or more optical transmitters that transmit light. The PPG system 435 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 435 may indicate the perfusion of blood in the illuminated region. For example, the PPG signal may indicate blood volume changes in the illuminated region caused by a user's pulse pressure. The processing module 430-a may sample the PPG signal and determine a user's pulse waveform based on the PPG signal. The processing module 430-a may determine a variety of physiological parameters based on the user's pulse waveform, such as a user's respiratory rate, heart rate, HRV, oxygen saturation, and other circulatory parameters.

In some implementations, the PPG system 435 may be configured as a reflective PPG system 435 where the optical receiver(s) receive transmitted light that is reflected through the region of the user's finger. In some implementations, the PPG system 435 may be configured as a transmissive PPG system 435 where the optical transmitter(s) and optical receiver(s) are arranged opposite to one another, such that light is transmitted directly through a portion of the user's finger to the optical receiver(s).

The number and ratio of transmitters and receivers included in the PPG system 435 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 435.

The PPG system 435 illustrated in FIG. 4 may include a reflective PPG system 435 in some implementations. In these implementations, the PPG system 435 may include a centrally located optical receiver (e.g., at the bottom of the wearable device 404) and two optical transmitters located on each side of the optical receiver. In this implementation, the PPG system 435 (e.g., optical receiver) may generate the PPG signal based on light received from one or both of the optical transmitters. In other implementations, other placements, combinations, and/or configurations of one or more optical transmitters and/or optical receivers are contemplated.

The processing module 430-a may control one or both of the optical transmitters to transmit light while sampling the PPG signal generated by the optical receiver. In some implementations, the processing module 430-a may cause the optical transmitter with the stronger received signal to transmit light while sampling the PPG signal generated by the optical receiver. For example, the selected optical transmitter may continuously emit light while the PPG signal is sampled at a sampling rate (e.g., 450 Hz).

Sampling the PPG signal generated by the PPG system 435 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 430-a may store the pulse waveform in memory 415 in some implementations. The processing module 430-a may process the pulse waveform as it is generated and/or from memory 415 to determine user physiological parameters described herein.

The processing module 430-a may determine the user's heart rate based on the pulse waveform. For example, the processing module 430-a may determine heart rate (e.g., in beats per minute) based on the time between peaks in the pulse waveform. The time between peaks may be referred to as an interbeat interval (IBI). The processing module 430-a may store the determined heart rate values and IBI values in memory 415.

The processing module 430-a may determine HRV over time. For example, the processing module 430-a may determine HRV based on the variation in the IBIs. The processing module 430-a may store the HRV values over time in the memory 415. Moreover, the processing module 430-a may determine the user's respiratory rate over time. For example, the processing module 430-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 430-a may store user respiratory rate values over time in the memory 415.

The wearable device 404 may include one or more motion sensors 445, such as one or more accelerometers (e.g., 6-D accelerometers) and/or one or more gyroscopes (gyros). The motion sensors 445 may generate motion signals that indicate motion of the sensors. For example, the wearable device 404 may include one or more accelerometers that generate acceleration signals that indicate acceleration of the accelerometers. As another example, the wearable device 404 may include one or more gyro sensors that generate gyro signals that indicate angular motion (e.g., angular velocity) and/or changes in orientation. The motion sensors 445 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 430-a may sample the motion signals at a sampling rate (e.g., 50 Hz) and determine the motion of the wearable device 404 based on the sampled motion signals. For example, the processing module 430-a may sample acceleration signals to determine acceleration of the wearable device 404. As another example, the processing module 430-a may sample a gyro signal to determine angular motion. In some implementations, the processing module 430-a may store motion data in memory 415. Motion data may include sampled motion data as well as motion data that is calculated based on the sampled motion signals (e.g., acceleration and angular values).

The wearable device 404 may store a variety of data described herein. For example, the wearable device 404 may store temperature data, such as raw sampled temperature data and calculated temperature data (e.g., average temperatures). As another example, the wearable device 404 may store PPG signal data, such as pulse waveforms and data calculated based on the pulse waveforms (e.g., heart rate values, IBI values, HRV values, and respiratory rate values). The wearable device 404 may also store motion data, such as sampled motion data that indicates linear and angular motion.

The wearable device 404, or other computing device, may calculate and store additional values based on the sampled/calculated physiological data. For example, the processing module 430 may calculate and store various metrics, such as sleep metrics (e.g., a Sleep Score), activity metrics, and readiness metrics. In some implementations, additional values/metrics may be referred to as “derived values.” The wearable device 404, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 404 is oriented on the user's finger and if the wearable device 404 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 430-a may compress the data stored in memory 415. For example, the processing module 430-a may delete sampled data after making calculations based on the sampled data. As another example, the processing module 430-a may average data over longer periods of time in order to reduce the number of stored values. In a specific example, if average temperatures for a user over one minute are stored in memory 415, the processing module 430-a may calculate average temperatures over a five minute time period for storage, and then subsequently erase the one minute average temperature data. The processing module 430-a may compress data based on a variety of factors, such as the total amount of used/available memory 415 and/or an elapsed time since the wearable device 404 last transmitted the data to the user device 406.

Although a user's physiological parameters may be measured by sensors included on a wearable device 404, other devices may measure a user's physiological parameters. For example, although a user's temperature may be measured by a temperature sensor 440 included in a wearable device 404, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 404 can make physiological measurements in a resting/sleep state in order to acquire cleaner physiological signals. In one example, the wearable device 404 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 404 may be configured to collect, store, and/or process data, and may transfer any of the data described herein to the user device 406 for storage and/or processing. In some aspects, the user device 406 includes a wearable application 450, an operating system (OS) 485, a web browser application (e.g., web browser 480), one or more additional applications, and a GUI 475. The user device 406 may further include other modules and components, including sensors, audio devices, haptic feedback devices, and the like. The wearable application 450 may include an example of an application (e.g., “app”) that may be installed on the user device 406. The wearable application 450 may be configured to acquire data from the wearable device 404, store the acquired data, and process the acquired data as described herein. For example, the wearable application 450 may include a user interface (UI) module 455, an acquisition module 460, a processing module 430-b, a communication module 420-b, and a storage module (e.g., database 465) configured to store application data.

In some cases, the wearable device 404 and the user device 406 may be included within (or make up) the same device. For example, in some cases, the wearable device 404 may be configured to execute the wearable application 450, and may be configured to display data via the GUI 475.

The various data processing operations described herein may be performed by the wearable device 404, the user device 406, the servers 410, or any combination thereof. For example, in some cases, data collected by the wearable device 404 may be pre-processed and transmitted to the user device 406. In this example, the user device 406 may perform some data processing operations on the received data, may transmit the data to the servers 410 for data processing, or both. For instance, in some cases, the user device 406 may perform processing operations that require relatively low processing power and/or operations that require a relatively low latency, whereas the user device 406 may transmit the data to the servers 410 for processing operations that require relatively high processing power and/or operations that may allow relatively higher latency.

In some aspects, the wearable device 404, user device 406, and server 410 of the system 400 may be configured to evaluate sleep patterns for a user. In particular, the respective components of the system 400 may be used to collect data from a user via the wearable device 404, and generate one or more scores (e.g., Sleep Score, Readiness Score) for the user based on the collected data. For example, as noted previously herein, the wearable device 404 of the system 400 may be worn by a user to collect data from the user, including temperature, heart rate, HRV, and the like. Data collected by the wearable device 404 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 404 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 400 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5 °C higher than the highest temperature during the longest period. In some aspects, the ring may measure a user's body temperature while the user is asleep, and the system 400 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 400 may support techniques for electric current-based feedback, which may be provided to a user of the wearable device 404 through generation of an electric current between two electrodes of the wearable device 404. The electric current may be generated according to a sequence of pulses which correspond to a type of feedback to be provided to the user.

That is, the wearable device 404 may have a capability to provide electric stimulation to a finger of a user as a form of feedback. Specifically, an inner housing of the wearable ring device may include an first electrode (e.g., an anode) and a second electrode (e.g., a cathode) such that when a user is wearing the wearable device 404, the wearable device 404 may be capable of passing an electrical current from the first electrode to the second electrode through the finger of the user that may be felt by the user as a form of electrical stimulation. In some examples, positions of the first electrode and the second electrode may be relative to a nerve in the finger of the user such that the electrical current may pass through the nerve. In such cases, electrical stimulation felt by the user may be felt along a length of the finger or in a fingertip.

The following provides an overview of aspects of the present disclosure:

Aspect 1: A wearable ring device, comprising: an outer housing; an inner housing; a set of electrodes positioned at least partially within the inner housing, the set of electrodes comprising at least a first electrode and a second electrode, wherein a position of the first electrode, a position of the second electrode, or both, are based at least in part on a size of the wearable ring device; and circuitry electrically coupled with the set of electrodes and configured to: generate a current between the first electrode and the second electrode via an electrical path external to the wearable ring device; and modulate a frequency of the current, wherein the frequency is configured for electric current-based haptic feedback.

Aspect 2: The wearable ring device of aspect 1, wherein, to modulate the frequency of the current, the circuitry is configured to modulate the frequency of the current in accordance with a first feedback pattern from a plurality of feedback patterns, wherein the first feedback pattern is associated with a first type of feedback.

Aspect 3: The wearable ring device of aspect 2, wherein the first feedback pattern defines the frequency of the current, a modulation duration, a modulation amplitude, a modulation pattern, a voltage of the current, or a combination thereof.

Aspect 4: The wearable ring device of any of aspects 2 through 3, wherein the circuitry is further configured to modulate the frequency of the current in accordance with a second feedback pattern from the plurality of feedback patterns, wherein the second feedback pattern is associated with a second type of feedback, and wherein the second feedback pattern is different from the first feedback pattern based at least in part on the second type of feedback being different from the first type of feedback and based at least in part on defining a second frequency of the current, a second modulation duration, a second modulation amplitude, a second modulation pattern, a second voltage of the current, or any combination thereof.

Aspect 5: The wearable ring device of any of aspects 1 through 4, further comprising: a second set of electrodes positioned at least partially within the outer housing, the second set of electrodes comprising at least a third electrode, wherein the circuitry is configured to: generate a second current between the third electrode and the first electrode, the second electrode, or both, via a second electrical path external to the wearable ring device, wherein the second electrical path is associated with measurement of physiological data.

Aspect 6: The wearable ring device of aspect 5, wherein the circuitry is configured to generate the current and the second current at different times.

Aspect 7: The wearable ring device of any of aspects 1 through 6, wherein the set of electrodes comprises a third electrode, and wherein the circuitry is configured to: generate a second current between the third electrode and either the first electrode or the second electrode via a second electrical path external to the wearable ring device; and generate a third current between the second electrode and the third electrode via a third electrical path external to the wearable ring device, wherein the electrical path, the second electrical path, and the third electrical path are different.

Aspect 8: The wearable ring device of any of aspects 1 through 7, wherein a voltage of the current is based at least in part on the size of the wearable ring device.

Aspect 9: The wearable ring device of any of aspects 1 through 8, wherein the electric current-based haptic feedback is biomimetic stimulation-based haptic feedback.

Aspect 10: A system for generation of haptic feedback, comprising: a wearable ring device comprising one or more sensors configured to measure physiological data from a user and comprising a set of electrodes configured to direct a current through a finger of the user, the set of electrodes comprising at least a first electrode and a second electrode; a user device communicatively coupled with the wearable ring device; and one or more processors communicatively coupled with the user device, the wearable ring device, or both, wherein the one or more processors are configured to: identify feedback to be provided to the user via the wearable ring device; select a first feedback pattern from a plurality of feedback patterns based at least in part on the first feedback pattern corresponding to the feedback to be provided to the user, wherein each feedback pattern of the plurality of feedback patterns is associated with a respective set of electrical parameters configured for electric current-based haptic feedback; and generate, based at least in part on identification of the feedback, a current between the first electrode and the second electrode in accordance with a first set of electrical parameters associated with the first feedback pattern.

Aspect 11: The system of aspect 10, wherein the one or more processors are further configured to identify second feedback to be provided to the user via the wearable ring device, the second feedback different from the feedback; select a second feedback pattern from the plurality of feedback patterns based at least in part on the second feedback pattern corresponding to the second feedback to be provided to the user, wherein the second feedback pattern is different than the first feedback pattern based at least in part on the second feedback being different than the feedback; and generate, based at least in part on identification of the second feedback, a second current between the first electrode and the second electrode in accordance with a second set of electrical parameters associated with the second feedback pattern of the plurality of feedback patterns, the second set of electrical parameters different from the first set of electrical parameters.

Aspect 12: The system of any of aspects 10 through 11, wherein the one or more processors are further configured to identify that the wearable ring device is positioned on a first finger of the user, wherein generating the current between the first electrode and the second electrode is based at least in part on the wearable ring device being positioned on the first finger.

Aspect 13: The system of aspect 12, wherein the set of electrodes comprise a third electrode, and wherein the one or more processors are further configured to select the first electrode and the second electrode from the set of electrodes based on the wearable ring device being positioned on the first finger of the user, wherein generating the current between the first electrode and the second electrode is based at least in part on selecting the first electrode and the second electrode from the set of electrodes.

Aspect 14: The system of any of aspects 12 through 13, wherein the one or more processors are configured to select the first electrode and the second electrode from the set of electrodes based at least in part on an electrical path between the first electrode and the second electrode being within a threshold proximity of a nerve of the first finger.

Aspect 15: The system of any of aspects 12 through 14, wherein the one or more processors are further configured to receive, via the user device, an indication that the wearable ring device is positioned on the first finger of the user, wherein the identification that the wearable ring device is positioned on the first finger of the user is based at least in part on the indication.

Aspect 16: The system of any of aspects 10 through 15, wherein the one or more processors are further configured to receive user feedback based at least in part on generating the current in accordance with the first set of electrical parameters associated with the first feedback pattern; and modify at least a subset of the first set of electrical parameters based at least in part on the user feedback.

Aspect 17: The system of aspect 16, wherein the user feedback is based at least in part on one or more user inputs, based at least in part on physiological data associated with the user in response to the current, or both.

Aspect 18: The system of any of aspects 16 through 17, wherein the electric current-based haptic feedback is biomimetic stimulation-based haptic feedback.

Aspect 19: The system of any of aspects 10 through 18, wherein the one or more processors are further configured to acquire physiological data from the user via the one or more sensors, wherein each respective set of electrical parameters is based at least in part on the physiological data.

Aspect 20: The system of any of aspects 10 through 19, wherein each respective set of electrical parameters comprises a frequency associated with the current, a modulation pattern of the frequency, a modulation duration, a modulation amplitude, a voltage of the current, or a combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an outer housing;
an inner housing;
a set of electrodes positioned at least partially within the inner housing, the set of electrodes comprising at least a first electrode and a second electrode, wherein a position of the first electrode, a position of the second electrode, or both, are based at least in part on a size of the wearable ring device; and
circuitry electrically coupled with the set of electrodes and configured to:
generate a current between the first electrode and the second electrode via an electrical path external to the wearable ring device; and
modulate a frequency of the current, wherein the frequency is configured for electric current-based haptic feedback.

2. The wearable ring device of claim 1, wherein, to modulate the frequency of the current, the circuitry is configured to:
modulate the frequency of the current in accordance with a first feedback pattern from a plurality of feedback patterns, wherein the first feedback pattern is associated with a first type of feedback.

3. The wearable ring device of claim 2, wherein the first feedback pattern defines the frequency of the current, a modulation duration, a modulation amplitude, a modulation pattern, a voltage of the current, or a combination thereof.

4. The wearable ring device of claim 2, wherein the circuitry is further configured to:
modulate the frequency of the current in accordance with a second feedback pattern from the plurality of feedback patterns, wherein the second feedback pattern is associated with a second type of feedback, and wherein the second feedback pattern is different from the first feedback pattern based at least in part on the second type of feedback being different from the first type of feedback and based at least in part on defining a second frequency of the current, a second modulation duration, a second modulation amplitude, a second modulation pattern, a second voltage of the current, or any combination thereof.

5. The wearable ring device of claim 1, further comprising:
a second set of electrodes positioned at least partially within the outer housing, the second set of electrodes comprising at least a third electrode, wherein the circuitry is configured to:
generate a second current between the third electrode and the first electrode, the second electrode, or both, via a second electrical path external to the wearable ring device, wherein the second electrical path is associated with measurement of physiological data.

6. The wearable ring device of claim 5, wherein the circuitry is configured to generate the current and the second current at different times.

7. The wearable ring device of claim 1, wherein the set of electrodes comprises a third electrode, and wherein the circuitry is configured to:
generate a second current between the third electrode and either the first electrode or the second electrode via a second electrical path external to the wearable ring device; and
generate a third current between the second electrode and the third electrode via a third electrical path external to the wearable ring device, wherein the electrical path, the second electrical path, and the third electrical path are different.

8. The wearable ring device of claim 1, wherein a voltage of the current is based at least in part on the size of the wearable ring device.

9. The wearable ring device of claim 1, wherein the electric current-based haptic feedback is biomimetic stimulation-based haptic feedback.

10. A system for generation of haptic feedback, comprising:
a wearable ring device comprising one or more sensors configured to measure physiological data from a user and comprising a set of electrodes configured to direct a current through a finger of the user, the set of electrodes comprising at least a first electrode and a second electrode;
a user device communicatively coupled with the wearable ring device; and
one or more processors communicatively coupled with the user device, the wearable ring device, or both, wherein the one or more processors are configured to:
identify feedback to be provided to the user via the wearable ring device;
select a first feedback pattern from a plurality of feedback patterns based at least in part on the first feedback pattern corresponding to the feedback to be provided to the user, wherein each feedback pattern of the plurality of feedback patterns is associated with a respective set of electrical parameters configured for electric current-based haptic feedback; and
generate, based at least in part on identification of the feedback, a current between the first electrode and the second electrode in accordance with a first set of electrical parameters associated with the first feedback pattern.

11. The system of claim 10, wherein the one or more processors are further configured to:
identify second feedback to be provided to the user via the wearable ring device, the second feedback different from the feedback;
select a second feedback pattern from the plurality of feedback patterns based at least in part on the second feedback pattern corresponding to the second feedback to be provided to the user, wherein the second feedback pattern is different than the first feedback pattern based at least in part on the second feedback being different than the feedback; and
generate, based at least in part on identification of the second feedback, a second current between the first electrode and the second electrode in accordance with a second set of electrical parameters associated with the second feedback pattern of the plurality of feedback patterns, the second set of electrical parameters different from the first set of electrical parameters.

12. The system of claim 10, wherein the one or more processors are further configured to:
identify that the wearable ring device is positioned on a first finger of the user, wherein generating the current between the first electrode and the second electrode is based at least in part on the wearable ring device being positioned on the first finger.

13. The system of claim 12, wherein the set of electrodes comprise a third electrode, and wherein the one or more processors are further configured to:
select the first electrode and the second electrode from the set of electrodes based on the wearable ring device being positioned on the first finger of the user, wherein generating the current between the first electrode and the second electrode is based at least in part on selecting the first electrode and the second electrode from the set of electrodes.

14. The system of claim 12, wherein the one or more processors are configured to select the first electrode and the second electrode from the set of electrodes based at least in part on an electrical path between the first electrode and the second electrode being within a threshold proximity of a nerve of the first finger.

15. The system of claim 12, wherein the one or more processors are further configured to:
receive, via the user device, an indication that the wearable ring device is positioned on the first finger of the user, wherein the identification that the wearable ring device is positioned on the first finger of the user is based at least in part on the indication.

16. The system of claim 10, wherein the one or more processors are further configured to:
receive user feedback based at least in part on generating the current in accordance with the first set of electrical parameters associated with the first feedback pattern; and
modify at least a subset of the first set of electrical parameters based at least in part on the user feedback.

17. The system of claim 16, wherein the user feedback is based at least in part on one or more user inputs, based at least in part on physiological data associated with the user in response to the current, or both.

18. The system of claim 10, wherein the one or more processors are further configured to:
acquire physiological data from the user via the one or more sensors, wherein each respective set of electrical parameters is based at least in part on the physiological data.

19. The system of claim 10, wherein each respective set of electrical parameters comprises a frequency associated with the current, a modulation pattern of the frequency, a modulation duration, a modulation amplitude, a voltage of the current, or a combination thereof.

20. The system of claim 16, wherein the electric current-based haptic feedback is biomimetic stimulation-based haptic feedback.
